(19) United States
(12) Patent Application Publication
(10) Pub. No.: US 2010/0298708 A1
(43) Pub. Date: Nov. 25, 2010

(54) BODY HEIGHT MEASURING APPARATUS

(76) Inventor: Weichao Pan, Guangdong (CN)

Correspondence Address: MOORE & VAN ALLEN PLLC, P.O. BOX 13706, Research Triangle Park, NC, 27709, US

(21) Appl. No.: 12/517,886

(22) PCT Filed: Dec. 13, 2007
(86) PCT No.: PCT/CN2007/003571

§ 371 Date: Jun. 5, 2009

(30) Foreign Application Priority Data
Dec. 22, 2006 (CN) 200620155199.6
Feb. 15, 2007 (CN) 200720048884.3

(51) Int. Cl. A61B 8/00

(52) U.S. Cl. 600/449

(57) ABSTRACT

A body height measuring apparatus is disclosed comprising a measuring bar, a ground mainframe, an ultrasound signal transmit unit and an ultrasound signal receive unit, a synchronous signal transmit unit and a synchronous signal receive unit. The first operational control unit and a second operational control unit synchronize the operation of the ultrasound signal transmit unit and the ultrasound signal receive unit through manipulation of the synchronous signal transmit unit and the synchronous signal receive unit, and calculate a height based on a transmission time during which an ultrasound signal travels from the ultrasound signal transmit unit to the ultrasound signal receive unit. As the body height is measured based on a unidirectional transmission of the ultrasound signal, the measurement error is reduced, the measuring accuracy is improved and an advantage in use is achieved.

### TECHNICAL FIELD

The present invention relates to a body height measuring apparatus, and particularly to an ultrasound body height measuring apparatus.

### BACKGROUND ART

An ultrasound body height measuring apparatus is characterized by high measurement accuracy and easy use. However, the existing ultrasound body height measuring apparatus is designed to use a measuring bar provided with both an ultrasound signal transmit device and an ultrasound signal receive device, and to calculate a value of body height based on a roundtrip time of an ultrasound signal. A disadvantage of such apparatus is in that if the ground is not sufficiently even, a reflection deviation of the ultrasound wave may occur, and there is a possibility the ultrasound wave is reflected by clothes during its transmission, rendering the measurement inaccurate.

In addition, existing ultrasound height measuring apparatus may provide the measuring bar with a rectangle ruler to enable the measuring bar to be placed horizontally upon transmission of an ultrasound signal. In use, the person to be measured is required to stand against a backrest, the measuring bar is then placed on the top of the head of the person and adjusted to be orthogonal to the backrest, then a switch is manually pressed to cause a measuring unit to transmit an ultrasound signal, and the height value is calculated once the measuring unit receives the signal. However, with such an apparatus the person must stand against a backrest, which limits the locations at which the apparatus can be used. Moreover, since such ultrasound measuring apparatus is operated manually, it is inconvenient to use.

### SUMMARY OF THE INVENTION

To overcome disadvantages in the existing techniques, the present invention provides a body height measuring apparatus with higher accuracy. To achieve the object of the present invention, the following technical solutions are employed.

According to an embodiment of the present invention, there is provided a body height measuring apparatus comprising: a measuring bar and a ground mainframe; an ultrasound signal transmit unit and an ultrasound signal receive unit, one disposed in the measuring bar and the other disposed in the ground mainframe; a synchronous signal transmit unit and a synchronous signal receive unit, one disposed in the measuring bar and the other disposed in the ground mainframe; and a first operational control unit and a second operational control unit disposed in the measuring bar and the ground mainframe respectively, to synchronize the operation of the ultrasound signal transmit unit and the ultrasound signal receive unit through manipulation of the synchronous signal transmit unit and the synchronous signal receive unit, and to calculate a height based on a transmission time during which an ultrasound signal travels from the ultrasound signal transmit unit to the ultrasound signal receive unit.

Optionally, the measuring bar comprises a horizontal tilt detection unit for detecting a tilt angle of the measuring bar, wherein the first operational control unit or the second operational control unit corrects a measured height based on the tilt angle of the measuring bar detected by the horizontal tilt detection unit.

Preferably, the synchronous signal receive unit comprises a plurality of synchronous signal receive points, wherein the first operational control unit or the second operational control unit corrects a measured height based on an offset distance between an actual receive point and a predetermined receive point.

More preferably, the plurality of synchronous signal receive points are arranged at equal intervals, the predetermined receive point is located in the center of a plurality of synchronous signal receive points, and the predetermined receive point corresponds to the synchronous signal receive point when the ultrasound signal transmit unit and the ultrasound signal receive unit are in the same vertical line.

More optionally, the ground mainframe is provided with a body weight measuring unit.

Further optionally, the measuring bar or the ground mainframe comprises a parameter input unit for inputting parameters of the person to be measured, the parameters comprising age and gender, and a body fat calculation unit for calculating body fat content based on a measurement of the body impedance between the two feet of the person and the input parameters.

Further optionally, the body height measuring apparatus according to an embodiment of the present invention comprises a display unit that is connected to the first operational control unit or the second operational control unit and used for displaying the measured result.

Further optionally, the measuring bar is provided with a contact switch for initiating the operation of the measuring bar.

Further optionally, the measuring bar is provided with a horizon detection unit for initiating the operation of the measuring bar once it determines that the measuring bar is horizontal.

Further preferably, the synchronous signal is an infrared signal, a radio signal or an optical signal.

Compared with existing ultrasound body height measuring apparatus, the apparatus according to an embodiment of the present invention has the following main advantages:

1. Eliminates the need of a reflection by allowing the measurement to be performed based on an unidirectional transmission of the ultrasound through use of synchronous signal to synchronize ultrasound transmitting and receiving. This greatly enhances the intensity and signal-to-noise ratio of the received signal, thus reducing error and avoiding interference caused by any intermediate obstacle.

2. Provides an error compensation by measuring the horizontal tilt angle, resulting in a faster and more convenient measurement with a higher accuracy;

3. The emission angle of the synchronous signal avoids an unreasonably large offset of the receive device of the measuring bar in the horizontal direction, which also improves the measuring accuracy.

### BRIEF DESCRIPTION OF DRAWINGS

The present invention will be further described below by way of specific embodiments in connection with the accompanying drawings, throughout which the same components are indicated by the same reference numbers.

FIG. 1 is a block diagram of a body height measuring apparatus according to an embodiment of the present invention;

FIG. 2 is a block diagram of a body height measuring apparatus according to another embodiment of the present invention;

FIG. 3 is a schematic diagram showing the correction of a measurement error caused by a tilt of the measuring bar according to an embodiment of the present invention;

FIG. 4 is a schematic diagram according to an embodiment of the present invention which includes a plurality of synchronous signal receive points;

FIG. 5 is a partial enlarged drawing at the point B of FIG. 4;

FIG. 6 is a block diagram of a body height measuring apparatus comprising a body weight measuring unit;

FIG. 7 is a block diagram of a body height measuring apparatus comprising a fat content calculation unit;

FIG. 8 is a block diagram of a body height measuring apparatus comprising a contact switch;

FIG. 9 is a block diagram of a body height measuring apparatus comprising a horizon detection unit;

FIG. 10 is a schematic diagram in which the synchronous signal and the ultrasound signal are both transmitted from top to bottom;

FIG. 11 is a schematic diagram in which the synchronous signal is transmitted from bottom to top and the ultrasound signal is transmitted from top to bottom;

FIG. 12 is a schematic diagram in which the synchronous signal and the ultrasound signal are both transmitted from bottom to top; and

FIG. 13 is a schematic diagram in which the synchronous signal is transmitted from top to bottom and the ultrasound signal is transmitted from bottom to top.

### DETAILED DESCRIPTION

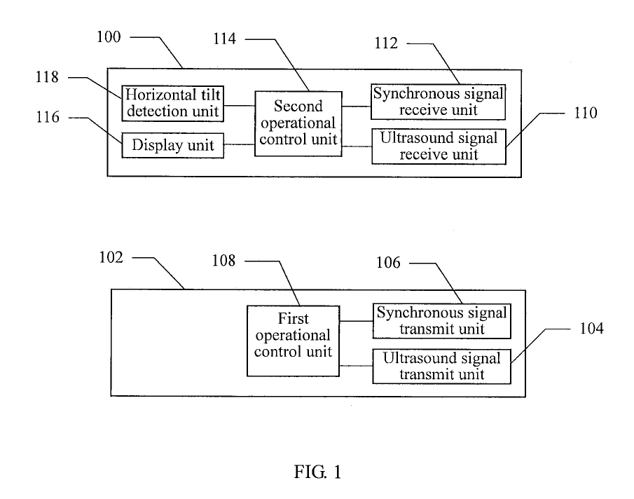

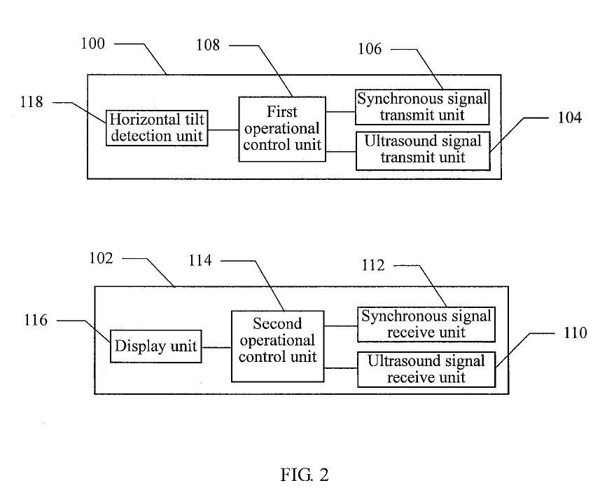

As shown in FIG. 1, the body height measuring apparatus according to one embodiment comprises a measuring bar 100 and a ground mainframe 102. The measuring bar 100 has an ultrasound signal receive unit 110, a synchronous signal receive unit 112 and a second operational control unit 114 mounted therein. The ground mainframe 102 has an ultrasound signal transmit unit 104, a synchronous signal transmit unit 106 and a first operational control unit 108 mounted therein. Alternatively, the ultrasound signal transmit unit 104, the synchronous signal transmit unit 106 and the first operational control unit 108 may be disposed in the measuring bar 100, and the ultrasound signal receive unit 110, the synchronous signal receive unit 112 and the second operational control unit 114 may be disposed in the ground mainframe 102, as shown in FIG. 2. In the embodiment, the synchronous signal may be an infrared signal, a radio signal or other optical signal.

During measurement, the measuring bar 100 is placed on the top of the head of a person to be measured. Under the control of the first operational control unit 108, the synchronous signal transmit unit 106 transmits a synchronous signal to the synchronous signal receive unit 112 in the measuring bar 100, meanwhile the ultrasound signal transmit unit 104 transmits an ultrasound signal to the ultrasound signal receive unit 110; the second operational control unit 114 synchronizes the operation of the ultrasound signal receive unit 110 with the ultrasound signal transmit unit 104 (i.e., to make them start operation at the same time) based on the synchronous signal received by the synchronous signal receive unit 112, and calculates a height of the person to be measured based on the time of delay (i.e., the period between the start of the ultrasound signal receive unit and the receipt of the ultrasound signal by the ultrasound signal receive unit) when the ultrasound signal receive unit 110 receives the ultrasound signal, in connection with the propagation velocity of the ultrasound wave in the air. That is, the height of the person is measured by calculating the distance that the ultrasound signal travels from the transmit unit 104 to the receive unit 110.

Optionally, the body height measuring apparatus according to an embodiment may also comprise a display unit 116 for displaying the measured result. The display unit 116 may be arranged on the measuring bar 100 or on the ground mainframe 102, as shown in FIG. 1 and FIG. 2.

Optionally, the body height measuring apparatus according to an embodiment may comprise a horizontal tilt detection unit 118 for detecting a tilt angle of the measuring bar, as shown in FIG. 1 and FIG. 2, wherein the second operational control unit 114 (or the first operational control unit 108) corrects a measured height based on the tilt angle of the measuring bar 100 detected by the horizontal tilt detection unit 118.

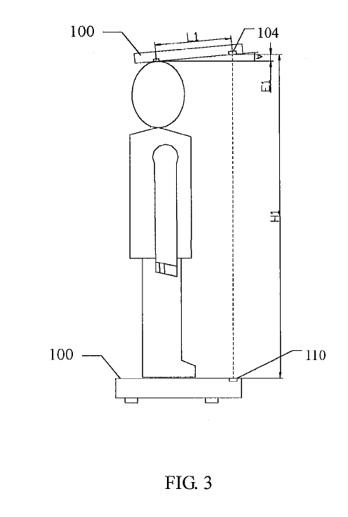

As shown in FIG. 3, the corrected actual height H0 is obtained by:

H0.H1.E1.H1.L1×sin.A.,

wherein L1 is the distance between the ultrasound signal transmit unit and the contact point of the top of the head of the person to be measured, A is tilt angle of the measuring bar, H1 is the measured height, and H0 is the corrected actual height.

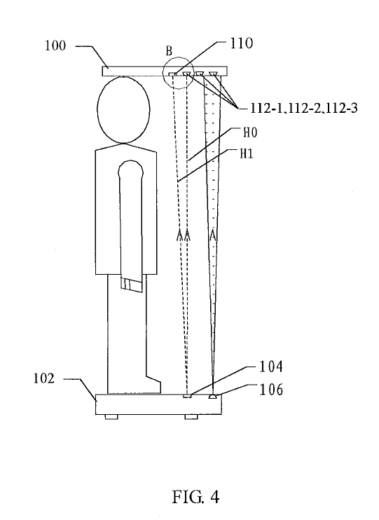

As shown in FIG. 4, the synchronous signal receive unit 112 of the body height measuring apparatus according to the embodiment may preferably comprise a plurality of receive points, based on which a measurement error caused by an offset of the measuring bar 100 in the horizontal direction may be corrected. As an example, the synchronous signal receive unit 112 comprises three receive points 112-1, 112-2 and 112-3, which are arranged in a row at equal intervals, with the interval being slightly larger than the width of the synchronous signal beam. In this example, the receive point 112-2 in the middle is the predetermined receive point, which corresponds to the synchronous receive point when the ultrasound signal transmit unit and the ultrasound signal receive unit are in the same vertical line.

As shown in FIG. 5, the offset distance L2 may be determined based on the distance between the receive point that actually receives the synchronous signal and the predetermined receive point. As can be seen, the measured height H1, the actual height H0 and the offset distance L2 form a right triangle. Therefore, the measurement error caused by the offset of the measuring bar 100 in the horizontal direction may be corrected as follows:

$${( H 1 )}^{2} = {( H 0 )}^{2} + {( L 2 )}^{2} , \\ H 0 = \sqrt{{( H 1 )}^{2} - {( L 2 )}^{2}} .$$

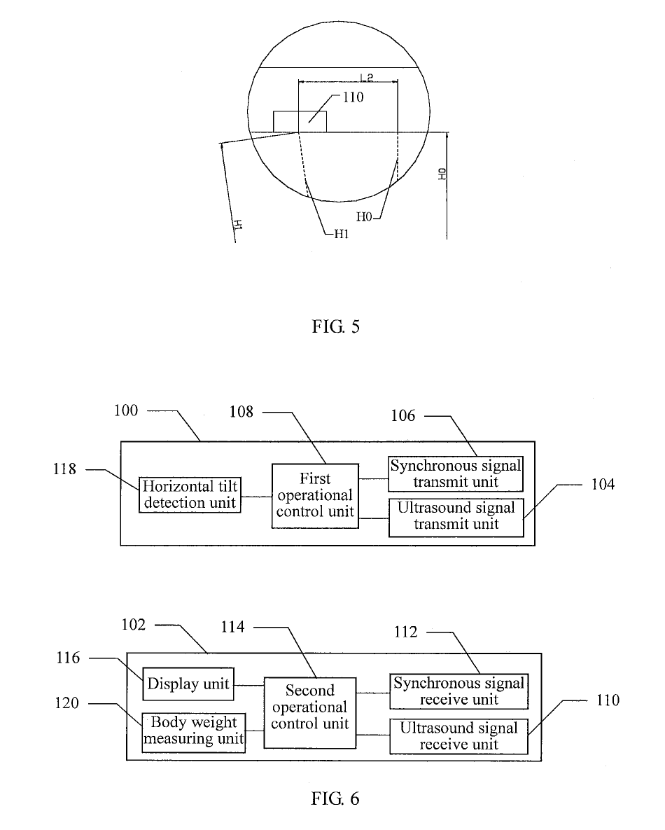

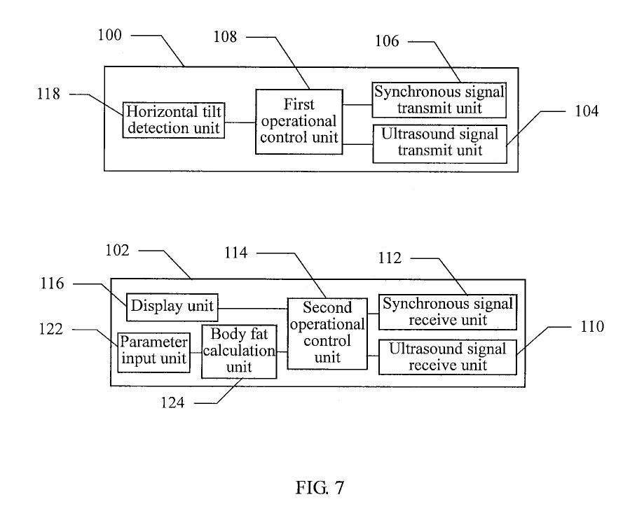

Still optionally, the ground mainframe 102 may further comprise a body weight measuring unit 120 to measure the body weight while measuring the height, and the measured result may be displayed on the display unit 116, as shown in FIG. 6.

Further optionally, the ground mainframe 102 may also comprise a parameter input unit 122 for inputting parameters of the person to be measured, the parameters comprising age and gender, and a body fat calculation unit 124 for calculating the body fat content based on a measurement of the body impedance between the two feet of the person and the input parameters, wherein the calculated results may be displayed on the display unit 116, as shown in FIG. 7.

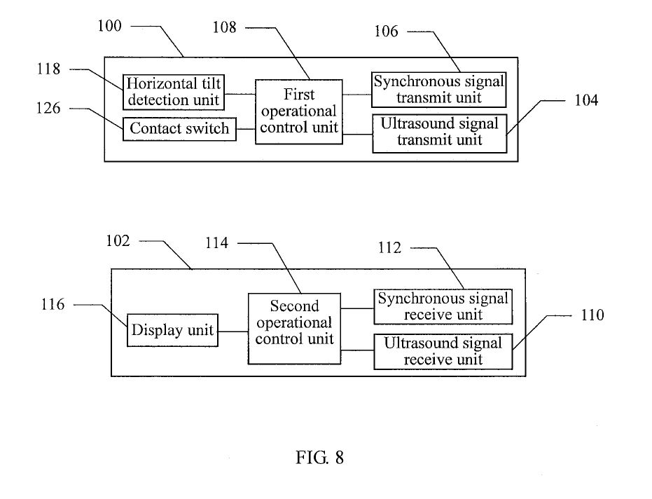

Further optionally, the measuring bar 100 may be provided with a contact switch 126. When the measuring bar 100 is placed on the top of the head of the person to be measured, the contact switch 126 is closed and the measurement is started, as shown in FIG. 8.

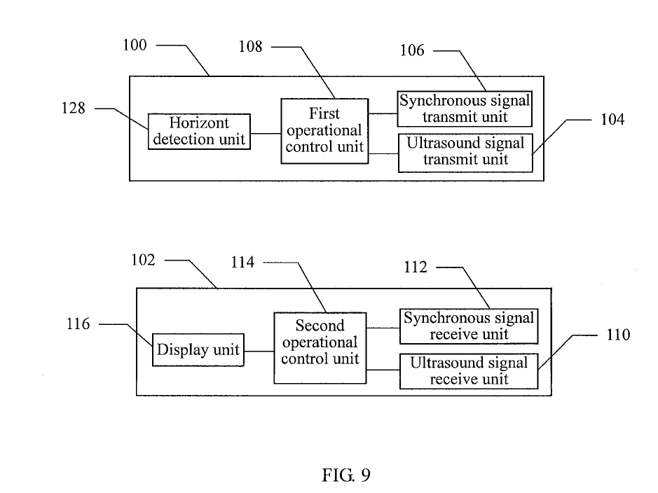

Further optionally, the measuring bar 100 may also comprise a horizon detection unit 128 for initiating the measurement once it determines that the measuring bar 100 is horizontal, as shown in FIG. 9.

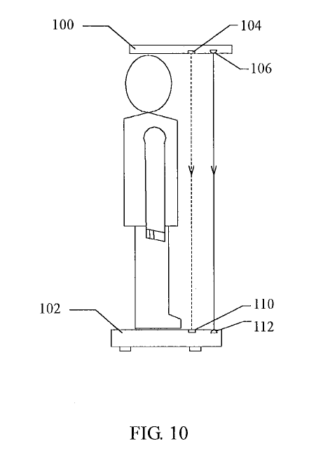

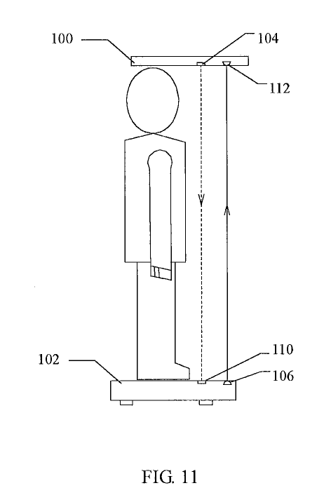

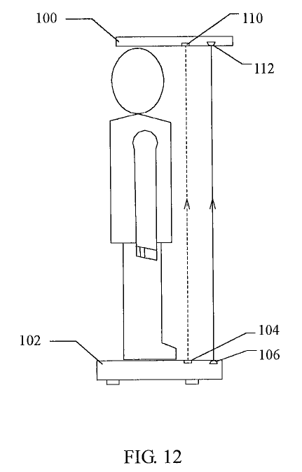

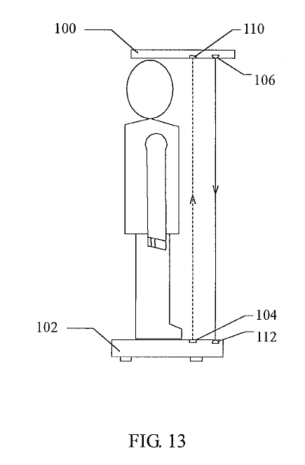

Furthermore, there are a number of choices as to the positions of the ultrasound signal transmit unit 104 and the ultrasound signal receive unit 110, the synchronous signal transmit unit 106 and the synchronous signal receive unit 112 of the body height measuring apparatus according to the embodiment. The ultrasound signal transmit unit 104 and the synchronous signal transmit unit 106 are disposed in the measuring bar 100, while the ultrasound signal receive unit 110 and the synchronous signal receive unit 112 are disposed in the ground mainframe 102, and thereby synchronous signals and ultrasound signals are transmitted from top to bottom, as shown in FIG. 10; the ultrasound signal transmit unit 104 and the synchronous signal receive unit 112 are disposed in the measuring bar 100, while the ultrasound signal receive unit 110 and the synchronous signal transmit unit 106 are disposed in the ground mainframe 102, and thereby synchronous signals are transmitted from bottom to top while ultrasound signals are transmitted from top to bottom, as shown in FIG. 11; the ultrasound signal transmit unit 104 and the synchronous signal transmit unit 106 are disposed in the ground mainframe 102, while the ultrasound signal receive unit 110 and the synchronous signal receive unit 112 are disposed in the measuring bar 100, and thereby synchronous signals and ultrasound signals are transmitted from bottom to top, as shown in FIG. 12; the ultrasound signal receive unit 110 and the synchronous signal transmit unit 106 are disposed in the measuring bar 100, while the ultrasound signal transmit unit 104 and the synchronous signal receive unit 112 are disposed in the ground mainframe 102, and thereby synchronous signals are transmitted from top to bottom while ultrasound signals are transmitted from bottom to top, as shown in FIG. 13.

Although the present invention has been described above with reference to specific embodiments thereof, the present invention is not limited to these specific embodiments. Instead, many modifications and variations may be made to the embodiments of the present invention. For example, the number of the synchronous signal receive points may be changed to 4, 5, 6, 7 and so on. In addition, such terms as “first”, “second”, “up” or “top”, “down” or “bottom”, and “horizontal” used in the description and the accompanying claims are intended to be illustrative rather than restrictive.

1-10. (canceled)

11. A body height measuring apparatus having a measuring bar and a ground mainframe, comprising:
an ultrasound signal transmit unit and an ultrasound signal receive unit, one being disposed in the measuring bar and the other being disposed in the ground mainframe;
a synchronous signal transmit unit and a synchronous signal receive unit, one being disposed in the measuring bar and the other being disposed in the ground mainframe; and
a first operational control unit and a second operational control unit, one being disposed in the measuring bar and the other being disposed in the ground mainframe, to synchronize the operation of the ultrasound signal transmit unit and the ultrasound signal receive unit through manipulation of the synchronous signal transmit unit and the synchronous signal receive unit, and to calculate a height based on a transmission time during which an ultrasound signal travels from the ultrasound signal transmit unit to the ultrasound signal receive unit.

12. The body height measuring apparatus according to claim 1,
wherein the measuring bar further comprises a horizontal tilt detection unit for detecting a tilt angle of the measuring bar;
wherein the first operational control unit or the second operational control unit corrects a measured height based on the tilt angle of the measuring bar detected by the horizontal tilt detection unit.

13. The body height measuring apparatus according to claim 2, wherein
the synchronous signal receive unit comprises a plurality of synchronous signal receive points; and
the first operational control unit or the second operational control unit corrects a measured height based on an offset distance between an actual receive point and a predetermined receive point.

14. The body height measuring apparatus according to claim 3, wherein the plurality of synchronous signal receive points are arranged at equal intervals, the predetermined receive point is located in the center of the plurality of synchronous signal receive points, and the predetermined receive point corresponds to the synchronous signal receive point when the ultrasound signal transmit unit and the ultrasound signal receive unit are in the same vertical line.

15. The body height measuring apparatus according to claim 1, wherein
the synchronous signal receive unit comprises a plurality of synchronous signal receive points; and
the first operational control unit or the second operational control unit corrects a measured height based on an offset distance between an actual receive point and a predetermined receive point.

16. The body height measuring apparatus according to claim 5, wherein the plurality of synchronous signal receive points are arranged at equal intervals, the predetermined receive point is located in the center of the plurality of synchronous signal receive points, and the predetermined receive point corresponds to the synchronous signal receive point when the ultrasound signal transmit unit and the ultrasound signal receive unit are in the same vertical line.

17. The body height measuring apparatus according to claim 1, wherein the ground mainframe further comprises a body weight measuring unit.

18. The body height measuring apparatus according to claim 1, wherein the measuring bar or the ground mainframe further comprises:
a parameter input unit for inputting parameters of the person to be measured, the parameters comprising age and gender; and
a body fat calculation unit for calculating body fat content based on a measurement of a body impedance between the two feet of the person to be measured and the input parameters.

19. The body height measuring apparatus according to claim 1, further comprising:
a display unit, connected to the first operational control unit or the second operational control unit, for displaying the measured result.

20. The body height measuring apparatus according to claim 1, wherein the measuring bar is provided with a contact switch for initiating the operation of the measuring bar.

21. The body height measuring apparatus according to claim 1, wherein the measuring bar is provided with a horizon detection unit for initiating the operation of the measuring bar once it determines that the measuring bar is horizontal.

22. The body height measuring apparatus according to claim 1, wherein the synchronous signal includes an infrared signal, a radio signal or an optical signal.
